(19) United States
(12) Patent Application Publication
(10) Pub. No.: US 2026/0354966 A1
(43) Pub. Date: Nov. 12, 2026

(54) PROVIDING PSEUDOPHAKIC PSEUDOACCOMMODATION USING WAVEFRONT MODIFIED INTRAOCULAR LENS

(71) Applicants: Marc Alan Michelson, Hoover, AL (US); Jennifer Leigh Michelson, Birmingham, AL (US)

(72) Inventors: Marc Alan Michelson, Hoover, AL (US); Jennifer Leigh Michelson, Birmingham, AL (US)

(21) Appl. No.: 19/672,897

(22) Filed: May 11, 2026

Related U.S. Application Data
(60) Provisional application No. 63/803,012, filed on May 9, 2025.

(51) Int. Cl. A61F 2/16; A61B 3/10

(52) U.S. Cl. CPC A61F 2/164; A61B 3/1015; A61F 2/1616; A61F 2240/001

(57) ABSTRACT

A method for enabling pseudoaccommodation in a pseudophakic eye may include evaluating a patient to identify higher order aberrations associated with an eye of the patient. A non-diffractive intraocular lens implant may be provided. The non-diffractive intraocular lens implant may be implanted in the eye of the patient to provide a resultant coma spread associated with the eye of greater than about 0.3 μm.

### CROSS-REFERENCE TO RELATED APPLICATIONS

This application claims the benefit of U.S. provisional patent application 63/803,012, entitled “INTRAOCULAR LENS DESIGN BASED ON INDUCED NEGATIVE VERTICAL COMA PAIRED WITH CORNEAL COMA TO CREATE PSEUDOACCOMMODATION,” filed on 9 May 2025, the entire disclosure of which is incorporated herein by reference.

### TECHNICAL FIELD

The present invention generally relates to eye lens replacement, and more particularly to wavefront modified intraocular lens for providing pseudoaccommodation.

### BACKGROUND

Modern cataract surgery routinely involves replacement of the natural crystalline lens with an artificially manufactured intraocular lens. An ideal artificial intraocular lens would be designed to replicate the functions of the natural human lens, allowing the eye to focus on any focal point in space providing full range of vision. The ability of the eye to see both distant objects and near objects by changing the focusing power from distance to near is called accommodation. Accommodation occurs when the natural lens changes shape altering the optical power of the eye. The precise dynamic fluidic movements of the lens are the result of stimulation of the ciliary body by the parasympathetic nervous system. Contraction or relaxation of the ciliary body not only changes the focus of the lens but also corrects for lens optical aberrations responsible for near vision. Over time, usually by the fifth decade of life, the lens slowly loses its ability to accommodate, and reading glasses become necessary to see near objects. The reduction of fluidity and ability of the lens to focus from distance to near is called presbyopia. As cataracts form, the natural lens not only becomes more dense and more opaque but also loses its fluidic characteristics. In order to completely replicate natural human vision, an intraocular lens should have the power to accommodate or simulate accommodation.

### SUMMARY

Consistent with an implementation, a method may include evaluating a patient to identify higher order aberrations associated with an eye of the patient. A non-diffractive intraocular lens implant may be provided, and the non-diffractive intraocular lens implant may be implanted in the eye of the patient to provide a resultant coma spread associated with the eye of greater than about 0.3 μm.

One or more of the following features may be included. Higher order aberrations may include at least third order Zernike terms. Evaluating the patient to identify higher order aberrations associated with the eye of the patient may include quantifying corneal coma of the eye. Higher order aberrations may include at least fourth order Zernike terms. Evaluating the patient to identify higher order aberrations associated with the eye of the patient may include quantifying corneal spherical aberration of the eye.

The non-diffractive intraocular lens implant may include a monofocal intraocular lens implant. The non-diffractive intraocular lens implant may include an aspheric intraocular lens implant. The non-diffractive intraocular lens implant may incorporate a 0.3 μm coma in one meridian. The non-diffractive intraocular lens implant may further incorporate 0.2 μm of negative spherical aberration.

Providing the non-diffractive intraocular lens implant may include selecting the non-diffractive intraocular lens having a coma based upon, at least in part, a corneal coma of the eye. Providing the non-diffractive intraocular lens implant may include providing the non-diffractive intraocular lens implant having a coma of 0.3 μm. The resultant coma spread may include a magnitude of a differences between a coma associated with the eye and a coma associated with the lens implant.

Implanting the non-diffractive intraocular lens implant may include orienting a coma associated with the non-diffractive intraocular lens implant relative to a coma associated with the eye to provide the resultant coma spread associated with the eye of greater than about 0.3 μm. Implanting the non-diffractive intraocular lens implant may include orienting a coma associated with the non-diffractive intraocular lens implant generally perpendicular to a coma associated with the eye. Implanting the non-diffractive intraocular lens implant may include intraoperatively evaluating an orientation of coma associated with the non-diffractive intraocular lens relative to a coma associated with the eye. Intraoperatively evaluating the orientation of coma associated with the non-diffractive intraocular lens implant relative to coma associated with the eye may include using intraocular aberrometry.

According to another implementation, a method may include evaluating a patient to identify higher order aberrations associated with an eye of the patient. The method may also include providing a non-diffractive intraocular lens implant configured to reduce a spherical aberration of the eye and generate negative vertical coma. The method may further include implanting the lens implant in the eye of the patient to provide a resultant coma spread associated with the eye of greater than about 0.3 μm.

One or more of the following features may be included. The non-diffractive intraocular lens implant may be a monofocal intraocular lens implant. The non-diffractive intraocular lens implant may incorporate 0.2 μm of negative spherical aberration. The non-diffractive intraocular lens implant may incorporate 0.3 μm of coma.

### BRIEF DESCRIPTION OF THE DRAWINGS

FIG. 1 diagrammatically depicts wavefront maps for individual Zernike aberrations including second order lower order aberrations and third and fourth order higher order aberrations, consistent with some embodiments.

FIG. 2 diagrammatically depicts wavefront maps of spherical aberrations and vertical coma, according to some embodiments.

FIG. 3 is a table of third and fourth order corneal Zernike aberrations and pseudoaccommodation with aspheric monofocal intraocular lenses, in a study consistent with the present disclosure.

FIG. 4 is a table of Root Mean Squared values of third and fourth order corneal Zernike aberrations and pseudoaccommodation with aspheric intraocular lenses, in a study consistent with the present disclosure.

FIG. 5 illustrates third order corneal higher order aberrations in a first eye of emmetropic patient following cataract surgery, consistent with the present disclosure.

FIG. 6 illustrates third order corneal higher order aberrations in a second eye of the emmetropic patient of FIG. 5, consistent with the present disclosure.

FIG. 7 depicts relationships between the magnitude of separation of paired terms for higher order aberrations and pseudoaccommodation, according to an experimental data set consistent with the present disclosure.

FIG. 8 diagrammatically depicts a non-diffractive intraocular lens implant coma orientation relative to a native corneal coma orientation, according to an example implementation.

FIG. 9 diagrammatically depicts wavefront deformation associated with corneal coma.

### DESCRIPTION OF EXAMPLE EMBODIMENTS

In general, the present disclosure may be directed toward providing patients multifocal vision in pseudophakic patients—i.e., patients having artificial intraocular lens implants in their eye. Consistent with some embodiments, such multifocal vision in pseudophakic patients may allow both distant vision as well as near vision without the need for glasses. Consistent with some embodiments, multifocal vision may be provided, and/or enabled, through the use of an intraocular lens incorporating higher order aberrations that compliment and/or interact with the intrinsic wavefront properties of the patient's cornea to produce wavefront patterns on the retinal that simulate and/or approximate the wavefront patterns in natural accommodation. Such simulation and/or approximation of wavefront patterns in natural accommodation may be termed pseudoaccommodation.

Consistent with some implementations of present disclosure, pseudoaccommodation may be achieved through the use of non-diffractive intraocular lenses. In general, intraocular lenses may be made of a solid material, such as acrylic, silicone, or other similar materials. Due to the materials used for intraocular lenses, these lenses are not capable of undergoing dynamic fluidic changes that replicate natural accommodation of the natural human lens. As such, using conventional techniques, in order to create multifocal vision, intraocular lenses generally rely on static properties of the artificial lens to mimic the accommodation process in order to treat presbyopia. As discussed above, the ability to see both near and distance with a non-fluid static intraocular lens, whether monofocal or multifocal, is called pseudoaccommodation.

Intraocular lenses can generally be divided into three main categories: monofocal lenses, multifocal lenses, and extended depth of focus lenses (EDOF). Monofocal intraocular lenses focus light from distant targets onto one focal point. In the emmetropic state, objects seen at a distance will focus light from distant objects onto the retina and be in sharp focus. By contrast, objects located in the near range will focus light behind the retina and the image will not be clearly in focus. For this reason, plus (+) lenses in reading glasses are required to move the focal point toward the retina, allowing near objects to be seen clearly. Accordingly, in conventional methodologies, monofocal intraocular lenses are not designed to treat or correct presbyopia because they only focus light at one single focal point.

Multifocal and EDOF intraocular lenses are designed to provide added depth of focus so an individual may see well both at distance, intermediate, and/or near after cataract surgery without the aid of glasses. Diffractive multifocal lenses are generally designed to diffract light into multiple focal points, allowing an individual to rapidly change the focus from near to distance. Multifocal intraocular lenses however are not universally tolerated. Multifocal intraocular lenses may produce high quality of vision at both distance and near, however not all patients achieve a satisfactory high-quality vision. For example, diffractive multifocal intraocular lenses may generate disturbing halos form light sources at night. Visual intolerance to diffractive multifocal intraocular lenses is not uncommon and therefore may not be suitable for many patients.

Additionally, diffractive multifocal intraocular lenses create multiple focal points so there becomes a progressive reduction of light energy at each of the numerous focal points as light traverses towards the retina. 100% of the light does not concentrate on the retina as each of the focal points reduces the intensity of light. Consequently, the ability to read in dim light may be compromised, and there may also be some loss in contrast sensitivity.

EDOF intraocular lenses are based on wavefront-shaping technology located on the anterior surface of the lens, typically in the central 2.2 mm region of the lens. Often, an EDOF lens may include two elements. The first element may include a slightly elevated plateau, and the second element may be a small curvature change across the entire 2.2 mm central optic. The lens may be designed to make parts of the emerging light energy wavefront advance, while the central portion of the wavefront may be delayed, resulting in a continuous extended focal range for near and distance vision. However, in practice, EDOF lenses may be more typically designed for intermediate and distant vision and not near vision. As such, EDOF lenses may not be reliably effective in providing simultaneous near and distance vision universally in all patients.

Frequently, EDOF and diffractive intraocular lens designs may be associated with higher levels of visual disturbances compared to monofocal intraocular lenses. Additionally, the lack of universal acceptance to multifocal lenses may be directly related to subtle variances in the way the cornea generates aberrations of light as light passes through, and interacts with, the cornea. Consistent with the present disclosure, it has been observed that aberrations of light generated by the cornea may influence the visual tolerance of a patient to diffractive intraocular lenses. Aberrated light rays deviating from the primary rays focusing on the retina can be measured and quantified. One common approach to measuring and quantifying the aberrations may include decomposing the aberrations into mathematical expressions called Zernike polynomials. As is generally known, each polynomial describes how much and what type of geometrical pattern each term contributes to the total optical wavefront aberration.

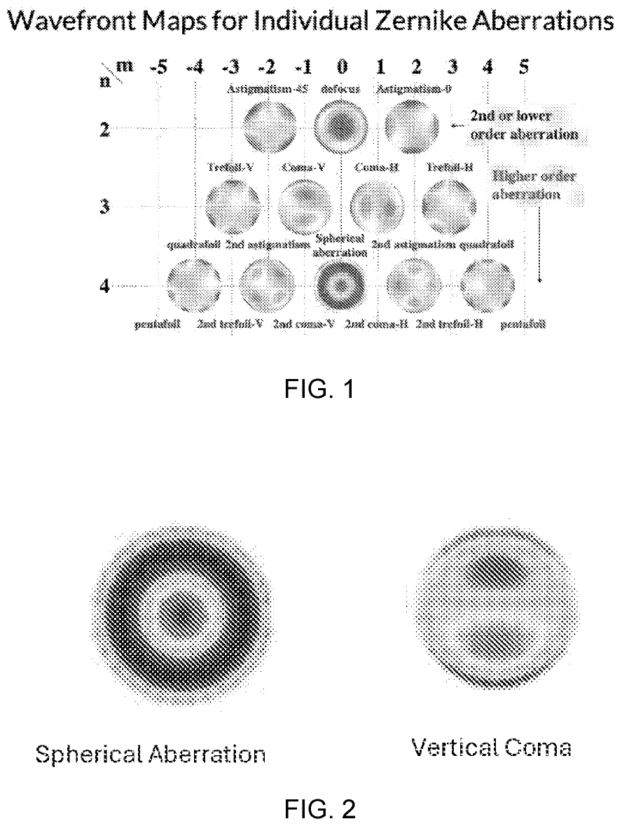

Zernike polynomials are a common tool for describing optical wavefronts aberrations. For example, and referring to FIG. 1, wavefront maps for individual Zernike aberrations are diagrammatically depicted, demonstrating wavefront maps for the individual Zernike aberrations, including the second order lower order aberrations and the third and fourth order higher order aberrations. The Zernike terms are generally grouped into two categories, lower order aberrations and higher aberrations. The lower order aberrations (e.g., Second order Zernike terms) can be corrected with glasses and include myopia, hyperopia, and astigmatism. The higher order aberrations (Third and Forth order Zernike terms) are more complex distortions of lights, including spherical aberrations, coma-like aberrations, and secondary/tertiary astigmatism, trefoil and quatrefoil. Higher order aberrations create complex distortions of light that cannot be corrected with glasses. Despite the fact that specific aberrations distort light and convolute the quality of an image, certain higher order aberrations are essential for high quality vision.

Every cornea may have a different wavefront pattern and corneal higher order aberrations vary from eye to eye, even in the same individual. The corneal wavefront pattern acts as a fingerprint of the cornea. Excessive aberrations in the combined Zernike third and fourth order may be directly associated with visual intolerance to a diffractive multifocal intraocular lens. RMS values of the combined Z₃ and Z₄ terms (i.e., Zernike third order polynomial and Zernike fourth order polynomial) in excess of 0.21 μm have been observed to be statistically significantly associated with increased visual dysphotopsias with diffractive multifocal intraocular lenses. Diffractive multifocal intraocular lenses may create phase induced aberrations as each focal point is created. If light passing through the cornea has a high RMS value with elevated Z₃ and Z₄ terms, these excessive aberrations may propagate and amplify through the multiple focal points. This may exaggerate the aberrations that are generated by the diffractive intraocular lens. Consequently, the image quality and contrast sensitivity may be reduced.

Accordingly, and consistent with the present disclosure, pseudoaccommodation may be achieve through the use of non-diffractive interocular lens implants (also variously referred to herein as non-diffractive IOLs, IOL implants, IOLs, and lens implants). In some implementations, non-diffractive IOLs may provide greater patient acceptance. Consistent with an implementation of the present disclosure, a method may include evaluating a patient to identify higher order aberrations associated with an eye of the patient. As noted above, higher order aberrations may include, but are not limited to, coma aberrations (third order aberrations) and spherical aberrations (fourth order aberrations). In some embodiments, as will be discussed in greater detail below, the magnitudes of the higher order aberrations of a patient's eye may be indicative of the likelihood of a patient achieving pseudoaccommodation consistent with the present disclosure. Further, consistent with some embodiments of the present disclosure, a non-diffractive intraocular lens implant may be provided. The non-diffractive intraocular lens implant may be implanted in the eye of the patient to provide a resultant coma spread associated with the eye of greater than about 0.3 μm. The resultant coma spread associated with the eye may include the wavefront pattern of the pseudophakic eye, resulting from any coma aberrations inherent in the eye of the patient and as modified by coma aberrations imparted by the IOL.

As noted above, conventional presbyopic intraocular lenses typically ignore the diversity and variability of wavefront patterns of the human cornea and their role in pseudoaccommodation. Consistent with the present disclosure, it has been observed that the wavefront characteristics of the cornea may play a critical role when predicting the ability of an eye to achieve a state of pseudoaccommodation. To better understand the role corneal higher order aberrations play in pseudoaccommodation, it requires an understanding of the changes of wavefront patterns that are induced by the human eye during physiologic accommodation. The natural lens also induces aberrations of light. During accommodation, as the shape of the natural lens changes its focus from distance to near very specific higher order optical aberrations have been observed to occur. Such higher order aberrations may include, for example, increased negative spherical aberration and increased negative vertical coma. As lens shape changes its focusing from distance to near, the values of these two higher order wavefront aberrations have been observed to increase. The elevation of these two higher order aberrations increases with increasing diopters of accommodation. FIG. 2 diagrammatically depicts wavefront maps of spherical aberration (fourth order aberrations) and vertical coma aberration (third order aberrations).

Consistent with the present disclosure, the use of specific higher order aberrations in an IOL may be capable of achieving pseudoaccommodation. Pseudoaccommodation may generally be defined as the ability of an eye with an intraocular lens to see distance and near based on static, not dynamic, qualities of the intraocular lens. In particular, the use of specific higher order aberrations in a non-diffractive IOL may be capable of achieving pseudoaccommodation. Such pseudoaccommodation may be achieve through change in the design of the intraocular lens and/or through the wavefront properties of the cornea. Consistent with the present disclosure, pseudoaccommodation may be achieved through the interaction of particular non-diffractive IOL design in conjunction with the wavefront properties of the cornea.

Accordingly, and as generally discussed above, in accordance with an implementation of the present disclosure, a method may include evaluating a patient to identify higher order aberrations associated with an eye of the patient. Accordingly, the wavefront properties of the cornea may be determined and/or quantified. It will be understood that in a patient undergoing, for example, lens replacement for the purpose of correcting cataracts, or similar procedures, both eyes may be evaluated. However, as noted above, each eye may produce different wavefront properties and/or produce different aberrations. As such, evaluating higher order aberrations associated with an eye of the patient may include evaluating higher order aberrations associated with each eye of the patient, respectively.

Evaluating and/or determining higher order aberrations associated with an eye of the patient may be accomplished through the use of aberrometry. In general, aberrometry may quantify the overall magnitude of wavefront deviations from an ideal wavefront. The wavefront deviations may be plotted in microns on the exit pupil and may be dependent on pupil size. Aberrometry may refer to measurement of the higher order aberrations. Aberrometers may be used to measure the ocular monochromatic higher order aberrations. In some implementations, the results of the measured ocular monochromatic higher order aberrations may be shown as an aberration map describing the variation in the optical path distance from the source to the retinal image through each point of the pupil. Consistent with some implementations, not only may the magnitude of each mode be considered, but also the possible combinations of the various modes. For example, certain combinations can change overall retinal image quality and, therefore, visual acuity. Many devices can measure ocular aberrations, including, but not limited to the following examples.

Hartmann-Shack aberrometer: A narrow laser beam is focused on the retina to generate a point source. The outcoming light rays experiencing all the ocular aberrations are split into multiple focal spots by a lenslet array on a CCD camera, with their displacement from the ideal used to reconstruct the wavefront. This approach may be less effective for highly irregular corneas such as keratoconus and post-surgical corneas. Examples of such aberrometers may include the Zywave aberrometer (Bausch+Lomb) and VISX WaveScan (AMO).

Tscherning aberrometer: A grid of laser spots is projected onto the retina, and a camera captures the distorted pattern to determine wavefront errors. This can work better in cases with highly irregular corneas but is less commonly used. Examples of such aberrometers may include the WaveLight Allegro Analyzer (Allegro).

Raytracing aberrometer: A thin laser beam is directed into the eye parallel to the visual axis. Each entrance point provides a projection on the retina, which is measured. A set of entrance points forms a set of projections, and a refraction map is reconstructed from these data. Such an aberrometer may work well in highly aberrated eyes. Examples of such aberrometers may include the iTrace (Tracey Technologies).

Dynamic sciascopy: Based on principles of time-based clinical retinoscopy. A projecting apparatus shines a scanning slit of light through a projecting lens and a set of mirrors. This light is reflected off the retina and passes through another lens (a receiving lens) and an aperture, ending on a set of photodetectors. Both the projecting and receiving systems rotate synchronously, allowing a comparison between the perceived light and the initially projected slit-shaped beams of light. Examples of such aberrometers include the OPD-Scan aberrometer (Nidek).

Anterior and posterior corneal surface mapping: the anterior and posterior corneal surfaces can be mapped by different machines, and Zernike polynomials can be calculated from this data. Examples include the Placido disc for anterior corneal surface, OCT for posterior corneal surface in the MS-39 (CSO), and Scheimpflug imaging for both corneal surfaces in the Pentacam (Oculus).

Through the use of aberrometry, higher order aberrations associated with the eye of the patient (including each eye individually, if the respective lenses of both eyes are to be replaced) may be evaluated. Consistent with various aberrometry systems and methods, the higher order aberrations of the total eye may be measured, and or the higher order aberrations associated with and/or resulting from the cornea may be measures. In some implementations, evaluating and/or determining higher order aberrations associated with an eye of the patient may include evaluating and/or determining higher order aberrations associated with and/or resulting from the cornea (i.e., corneal higher order aberrations). It will be appreciated that in some embodiments, higher order aberrations associated with and/or resulting from the total eye could also be continued.

Consistent with the present disclosure, higher order aberrations may include at least third order Zernike terms. Accordingly, evaluating the patient to identify higher order aberrations associated with the eye of the patient may include quantifying corneal coma of the eye. Additionally, higher order aberrations may include at least fourth order Zernike terms. Accordingly, evaluating the patient to identify higher order aberrations associated with the eye of the patient may include quantifying corneal spherical aberration of the eye.

As will be generally discussed in greater detail below, in some implementations evaluating the patient to identify higher order aberrations associated with the eye of the patient may be utilized, at least in part, to determine the suitability of a patient for intraocular lens replacement consistent with the present disclosure, and/or the likelihood that the patient will achieve pseudoaccommodation consistent with the present disclosure. Additionally and/or alternatively, evaluating the patient to identify higher order aberrations associated with the eye of the patient may quantify certain higher order aberrations, such as spherical aberrations and coma aberrations associated with the eye of the patient (i.e., higher order aberrations produced by the cornea of the eye of the patient). Quantifying such higher order aberrations of the eye of the patient may be utilized, at least in part, for providing a suitable intraocular lens (e.g., one or more of producing a suitable intraocular lens and selecting a suitable intraocular lens), as discussed below in greater detail.

Consistent with the present disclosure, a non-diffractive intraocular lens implant may be provided. Further, the non-diffractive intraocular lens implant may be implanted in the eye of the patient to provide a resultant coma spread associated with the eye of greater than about 0.3 μm. As used herein, a resultant coma spread associated with the eye of greater than about 0.3 μm may include the resultant coma spread associated with the pseudophakic eye, taking into consideration the aberrations introduced by the native cornea and as modified by the non-diffractive intraocular lens implant. The resultant coma spread may include a magnitude of a difference between a coma associated with the eye and a coma associated with the lens implant.

As noted above, many patients may not adequately tolerate diffractive intraocular lenses. Accordingly, consistent with the present disclosure, a non-diffractive intraocular lens implant may be implemented. Further, consistent with the present disclosure pseudoaccommodation may be achieve using the non-diffractive intraocular lens, thereby providing both distant and near vision in patients that may not tolerate conventional multifocal intraocular lenses, and/or without the need for multifocal intraocular lenses. Consistent with some embodiments of the present disclosure, the non-diffractive intraocular lens implant may include a monofocal intraocular lens implant.

Consistent with some implementation, the present disclosure may increase the negative vertical coma of the eye of the patient. For example, in some instances it has been observed that a relatively larger spread between negative vertical coma and positive coma may predict a greater likelihood of the patient achieving pseudoaccommodation, thereby having multifocal vision using a monofocal intraocular lens. In some instances, for patients that may not have a large coma spread in an eye, consistent with some embodiments the present disclosure may allow coma to be added to the IOL to increase the coma spread. In some such embodiment, the increase in coma spread provided by the IOL may allow the patient to see near and distance.

As noted above, the non-diffractive intraocular implant may be implanted to provide a resultant coma spread associated with the eye of greater than about 0.3 μm. As such, in some embodiments, providing the non-diffractive intraocular lens implant may include selecting the non-diffractive intraocular lens having a coma based upon, at least in part, a corneal coma of the eye. Selecting the non-diffractive intraocular lens may include producing a custom IOL have a desired coma and/or selecting an IOL having a desired coma from a plurality of pre-manufactured IOLs having different coma values (e.g., from a set of IOLs having different coma values and/or from a range of IOLs having different coma values).

As noted above, the resultant coma may include the coma of the native cornea as modified by the coma of the IOL. As such, for example, if an eye of a patient has a coma of 0.2 μm, an IOL having a coma of 0.1 μm may be selected to provide a resultant coma of 0.3 μm. However, in some instances, a high coma may be desirable for achieving pseudoaccommodation. Accordingly, for example, if an eye of a patient has a coma of 0.2 μm or 0.3 μm, an IOL having a coma of 0.3 μm may be selected to provide a resultant coma of 0.5 μm or 0.6 μm. Additionally, even if an eye of a patient does not have coma, and IOL having a coma of 0.3 μm may be selected to provide a resultant coma spread of 0.3 μm, which may provide adequate pseudoaccommodation. In some such implementations, selecting an IOL may include selecting an IOL having a coma of 0.3 μm or greater. For example, the non-diffractive intraocular lens implant may incorporate a 0.3 μm coma in one meridian. However, as noted, the non-diffractive intraocular lens may incorporate other coma values to achieve a resultant 0.3 μm coma, depending upon the native coma of the cornea of the eye of the patient. Therefore, in some embodiments, providing the non-diffractive intraocular lens implant may include providing the non-diffractive intraocular lens implant having a coma of at least 0.3 μm.

Consistent with some implementations, in addition to coma, the non-diffractive intraocular lens implant may also incorporate other higher order aberrations. For example, consistent with the present disclosure it has been identified that spherical aberration may facilitate pseudoaccommodation. For example, in some instances monofocal intraocular lenses have changed from a “spherical” lens design to an “aspheric” lens design. In some cases, aspheric lenses may reduce the effect of the corneal positive spherical aberration by adding negative spherical aberration to the intraocular lens. For some patients, this may improve night vision and/or may improve contrast sensitivity. Consistent with the present disclosure, it has been determined that in some cases aspheric intraocular lens design may increase the incidence of pseudoaccommodation. For example, consistent with the present disclosure, an evaluation of patients implanted with aspheric monofocal intraocular lenses observed an increase in the incidence of pseudoaccommodation in proportion to increasing amounts of vertical coma and reduced amounts of positive spherical aberrations present in the cornea. Consistent with this study, corneal wavefront properties were analyzed comparing eyes with the ability to, or not to pseudoaccommodate. Third and fourth order Zernike terms were measured utilizing the Hurnphry Atlas/Vol CT Program. The absolute value for each term as well as the Root Mean Square (RMS) and values of paired terms were analyzed. 55 eyes were studied. 25 eyes had the ability to pseudoaccommodate and 30 were not able to see near without reading glasses.

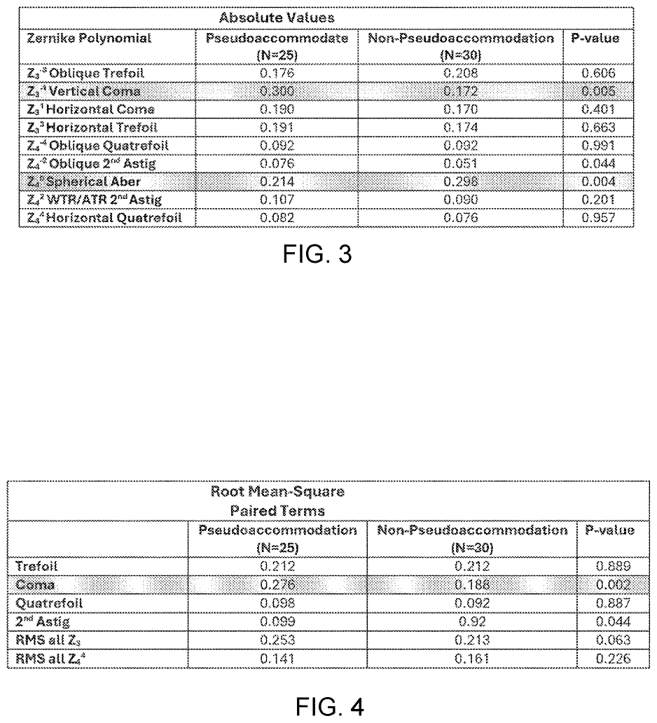

With reference also to FIG. 3, higher absolute values of corneal negative vertical coma (Z₃⁻¹ p=0.005) and lower absolute values of corneal spherical aberration (Z₄^{o} p=0.004) were statistically significantly correlated to pseudoaccommodation in an eye an aspheric monofocal intraocular lens.

Referring also to FIG. 4, during the study the root mean squared value of the paired Zernike terms horizontal coma (Z₃¹) and vertical coma (Z₃⁻¹) were found to be statistically significantly correlated to pseudoaccommodation in eyes with an aspheric monofocal intraocular lens. Aspheric monofocal intraocular lens designs with 0.2 μm negative spherical aberration are believed to contribute to the total wavefront by additionally reducing spherical aberration explaining the observation of increased incidence of pseudoaccommodation with aspheric monofocal intraocular lenses. The corneal wavefront patterns with tower corneal spherical aberration and increased vertical coma in eyes with aspheric monofocal intraocular lenses that pseudoaccommodate is understood to parallel and/or mimic the same wavefront patterns in lens induced natural accommodation in phakic eyes.

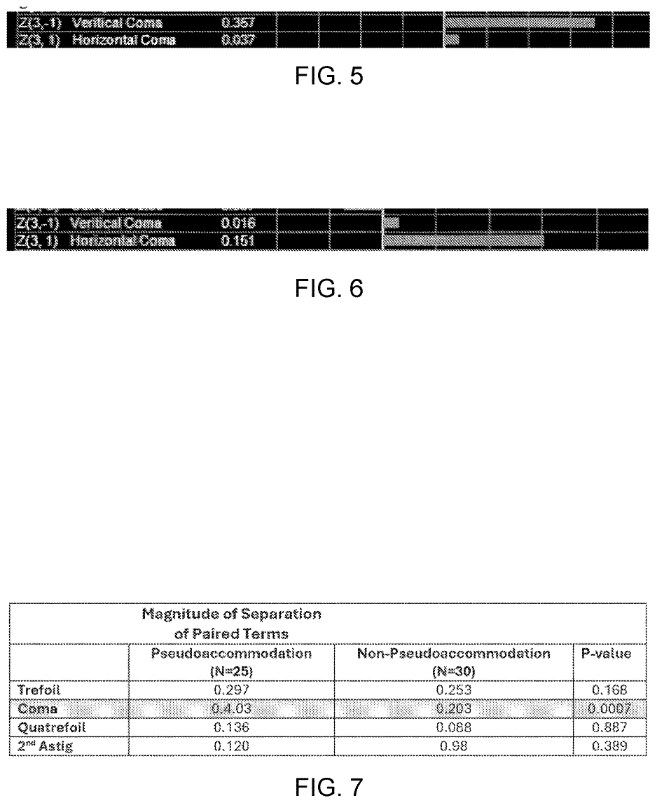

Referring also to FIGS. 5-6 an observational study of an example patient following cataract surgery with an aspheric monofocal intraocular lens who is emmetropic with 20/20 distance and perfect reading vision (J1) near without correction in one eye. As shown in FIG. 5, the third order corneal higher order aberrations are displayed. The vertical coma value is 0.357 μm and pseudoaccommodation is present. By contrast, FIG. 6 shows the corneal coma values in the opposite eye of the same patient who has 20/20 vision at distance but is not able to see well at near (J5). The vertical coma value is 0.016 μm and pseudoaccommodation is absent. Based on, at least in part, the foregoing, the corneal wavefront patterns are understood to significantly influence the ability of an eye to pseudoaccommodate in an eye with an aspheric monofocal intraocular lens.

In consideration of the foregoing studies and observations, consistent with some implementations, the non-diffractive intraocular lens implant may include an aspheric intraocular lens implant. For example, in some implementations, the non-diffractive intraocular lens implant may further incorporate 0.2 μm of negative spherical aberration. For example, the natural accommodation physiologic process may induce negative spherical aberration and negative vertical coma. When pseudoaccommodation occurs with an aspheric monofocal IOL, the increased negative spherical aberration from the IOL and the vertical coma from the cornea may mimic the wavefront pattern that occurs in accommodation allowing the eye to seen both distance and near without utilizing diffractive optics.

Aspheric intraocular lenses introduce negative spherical aberration into to optical system of the eye designed to counter the natural positive spherical aberration of the cornea improving contrast sensitivity to improve night vision. The negative asphericity of the intraocular lens by happenstance may also be contributing to the eye's ability to generate pseudoaccommodation in eyes where higher values of coma are present. The aspheric lens design in combination with higher corneal coma values may enhance the pseudoaccommodation in some embodiments.

Consistent with some implementations, implanting the non-diffractive intraocular lens implant may include orienting a coma associated with the non-diffractive intraocular lens implant relative to a coma associated with the eye to provide the resultant coma spread associated with the eye of greater than about 0.3 μm. For example, as generally discussed above, corneal coma values have been linked to pseudoaccommodation. However consistent with the present disclosure that is another aspect of corneal coma that has never been recognized. Coma may have a negative or positive value. In addition, coma may have a vertical or horizontal position. The magnitude of the difference between values of the corneal horizontal coma and corneal vertical coma may be extremely significant when predicting pseudoaccommodation with an aspheric monofocal intraocular lens. The value of this separation may be significant to the point that 0.3 μm to 0.4 μm differential between vertical and horizontal coma values may, in some instances, reliably predict pseudoaccommodation in an eye with an aspheric monofocal intraocular lens (p value=0.0007). This correlation is shown, for example in the experimental sample data depicted in FIG. 7. This means the greater the absolute value between horizontal and vertical coma, the greater may be ability to generate near vision in an emmetropic aspheric pseudophakic eye.

Consistent with the present disclosure, orienting the coma associated with the non-diffractive intraocular lens implant may include positioning IOL with respect to the eye, specifically the coma of the native eye, to achieve the resultant coma. The orientation may include any translational or rotational positioning of the coma of the IOL with respect to the native coma of the eye. Non-diffractive intraocular lens implant selection may play a part in this, for example by selecting the IOL having desired coma characteristics. In some implementations, the non-diffractive intraocular lens implant may be positioned to maximize the difference between the coma of the IOL and the pre-existing corneal coma values. For example, the coma portion of the IOL may be positioned inferiorly or horizontally depending on existing corneal coma (whether it be horizontal or vertical). A desired position of the coma portion of the IOL may be predetermined, e.g., by analyzing corneal coma values preoperatively.

Consistent with the present disclosure, a non-diffractive presbyopia-correcting IOL design and implantation may be achieved that may amplify the corneal higher order aberrations of each individual cornea to simulate the wavefront pattern induced by the natural lens during accommodation. Such a design and implementation may eliminate and/or reduce the visual halos and dysphotopsias associated with a diffractive or EDOF intraocular lenses, and may provide sufficient pseudoaccommodation to create acceptable, if not excellent, near and distance vision.

Consistent with some implementations of the present disclosure, a monofocal non-diffractive lens design and/or implementation (e.g., via positioning and implantation) may generate multifocal vision based on the variability of and the influence of the IOL on the optical aberrations generated by the cornea. The design and/or implementation of the IOL may, in some embodiments, incorporate 0.2 μm of negative spherical aberration. In addition, in some embodiments, the IOL may incorporate 0.3 μm of coma in one meridian. An aspheric IOL design incorporating 0.3 μm of coma may be implanted and positioned to maximize the separation of values of pre-existing corneal coma. Corneas can be categorized into patterns of low or elevated coma values. Pre-existing coma values may be analyzed preoperatively. By positioning the IOL to achieve the greatest separation of coma values between the IOL and the cornea, pseudoaccommodation may be achieved without, or with reduced levels of, the unwanted visual side effects of a multifocal intraocular lens. This new IOL design and/or implementation may allow a surgeon to pre-operatively accurately distinguish appropriate patient populations to achieve multifocality and pseudoaccommodation. Positioning a coma wavefront modified IOL may be similar to positioning a toric intraocular lens using intraocular aberrometry.

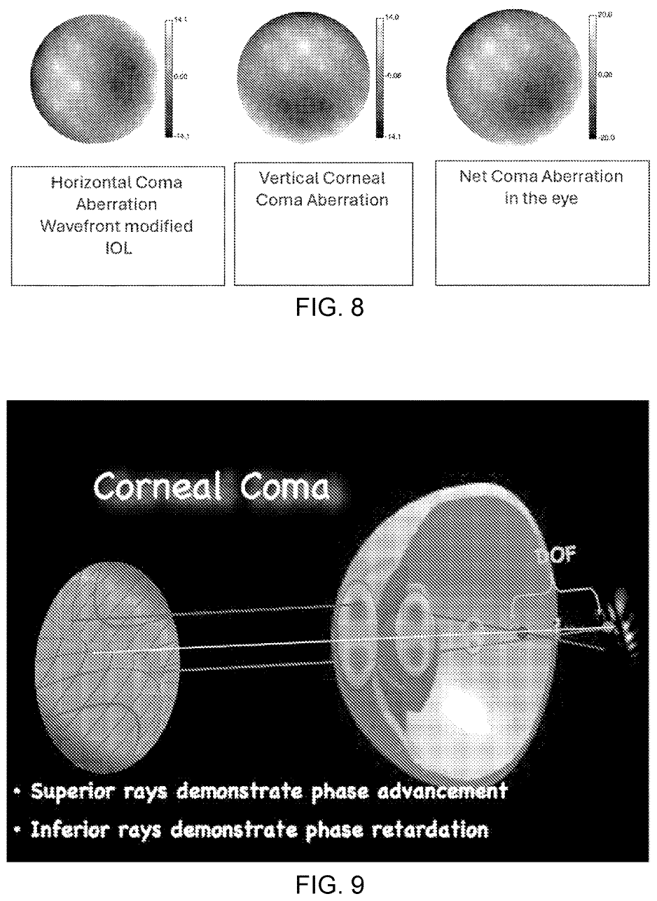

As generally noted above, implanting the non-diffractive intraocular lens implant may include orienting a coma associated with the non-diffractive intraocular lens implant generally perpendicular to a coma associated with the eye. As also generally discussed above, implanting the non-diffractive intraocular lens implant may include preoperatively evaluating coma of eye, and using this information along with the known information regarding the coma of the IOL to position the coma portion of the IOL perpendicularly relative to the coma of the eye. For example, and with reference also to FIG. 8, an example embodiment is depicted in which an eye may have a native vertical corneal coma aberration. A wavefront modified non-diffractive intraocular lens may be selected and positioned having a horizontal coma aberration.

Consistent with some implementations, implanting the non-diffractive intraocular lens implant may include intraoperatively evaluating an orientation of coma associated with the non-diffractive intraocular lens relative to a coma associated with the eye. For example, intraoperatively evaluating the orientation of coma associated with the non-diffractive intraocular lens implant relative to coma associated with the eye may include using intraocular aberrometry. Any suitable intraocular aberrometry techniques and systems may be utilized, including, but not limited to those disclosed hereinabove.

Consistent with some embodiments of the present disclosure, a wavefront monofocal intraocular lens design and/or implementation may be capable of achieving pseudophakic pseudoaccommodation in corneas with low vertical coma values. The design and/or implementation may incorporate Zernike third order wavefront of 0.3 μm of horizontal coma as well as 0.2 μm of negative spherical aberration. The designated coma portion of the IOL may be implanted and positioned either inferiorly or horizontally depending on the existing corneal coma, whether it be horizontal or vertical. The positioning of the coma component of the IOL may be predetermined by analyzing the corneal coma values preoperatively. Accordingly, in some embodiments, the IOL may be positioned to maximize the maximum difference between the coma of the IOL and the pre-existing corneal coma values.

In some embodiments, a wavefront modified IOL may be designed and/or implemented to match the wavefront properties of the cornea to achieve multifocality without the use of diffractive optics on the lens design. Consistent with some implementations, intraoperative aberrometry may be incorporated to accurately position the IOL in the eye to maximize the vertical and horizontal coma differential.

The deformation of a wavefront that produces coma aberration results in a portion of light focusing in front of the retina and another portion of light focusing behind the retina. If the medial focal point is on the retinal, then the separation of paraxial focal point may create an increased depth of field, which may allow an eye to pseudoaccommodate to see distance and near, as generally depicted in FIG. 9.

Accordingly, consistent with some aspect of the present disclosure, IOL designs and methods may be provided that may result in obtaining and/or providing a non-diffractive intraocular lens implant that may provide the eye with increased coma aberrations. According to some aspects, the present disclosure may provide methods of obtaining and/or providing an IOL capable of reducing the spherical aberration of the eye. According to some aspects, the present disclosure may provide methods of obtaining and/or providing an IOL capable of complementing the optical aberrations from the corneal surfaces to increase the net difference between the horizontal and vertical coma values. According to some aspects, the present disclosure may provide an IOL which may be capable of creating a wavefront pattern that mimics the wavefront patterns generated by the natural accommodating process without disturbing the central optics of the IOL with diffraction or wavefront stretching techniques and without diminution of light focusing on the retina.

While various example embodiments, implementations and possible features and advantages have been described herein, such description is provided for the purpose of explaining various aspects of the disclosed subject matter. As such, the description of such should not be considered a limitation on the present invention, which should be given the fully breadth of the claims appended hereto.

What is claimed is:

1. A method comprising:
evaluating a patient to identify higher order aberrations associated with an eye of the patient;
providing a non-diffractive intraocular lens implant; and
implanting the non-diffractive intraocular lens implant in the eye of the patient to provide a resultant coma spread associated with the eye of greater than about 0.3 μm.

2. The method according to claim 1, wherein higher order aberrations include at least third order Zernike terms.

3. The method according to claim 2, wherein evaluating the patient to identify higher order aberrations associated with the eye of the patient include quantifying corneal coma of the eye.

4. The method according to claim 1, wherein higher order aberrations include at least fourth order Zernike terms.

5. The method according to claim 4, wherein evaluating the patient to identify higher order aberrations associated with the eye of the patient include quantifying corneal spherical aberration of the eye.

6. The method according to claim 1, wherein the non-diffractive intraocular lens implant includes a monofocal intraocular lens implant.

7. The method according to claim 1, wherein the non-diffractive intraocular lens implant includes an aspheric intraocular lens implant.

8. The method according to claim 1, wherein the non-diffractive intraocular lens implant incorporates a 0.3 μm coma in one meridian.

9. The method according to claim 1, wherein the non-diffractive intraocular lens implant further incorporates 0.2 μm of negative spherical aberration.

10. The method according to claim 1, wherein providing the non-diffractive intraocular lens implant comprises selecting the non-diffractive intraocular lens having a coma based upon, at least in part, a corneal coma of the eye.

11. The method according to claim 1, wherein providing the non-diffractive intraocular lens implant comprises providing the non-diffractive intraocular lens implant having a coma of at least 0.3 μm.

12. The method according to claim 1, wherein the resultant coma spread comprises a magnitude of a differences between a coma associated with the eye and a coma associated with the lens implant.

13. The method according to claim 1, wherein implanting the non-diffractive intraocular lens implant includes orienting a coma associated with the non-diffractive intraocular lens implant relative to a coma associated with the eye to provide the resultant coma spread associated with the eye of greater than about 0.3 μm.

14. The method according to claim 1, wherein implanting the non-diffractive intraocular lens implant includes orienting a coma associated with the non-diffractive intraocular lens implant generally perpendicular to a coma associated with the eye.

15. The method according to claim 1, wherein implanting the non-diffractive intraocular lens implant includes intraoperatively evaluating an orientation of coma associated with the non-diffractive intraocular lens relative to a coma associated with the eye.

16. The method according to claim 15, wherein intraoperatively evaluating the orientation of coma associated with the non-diffractive intraocular lens implant relative to coma associated with the eye includes using intraocular aberrometry.

17. A method comprising:
evaluating a patient to identify higher order aberrations associated with an eye of the patient;
providing a non-diffractive intraocular lens implant configured to reduce a spherical aberration of the eye and generate negative vertical coma; and
implanting the lens implant in the eye of the patient to provide a resultant coma spread associated with the eye of greater than about 0.3 μm.

18. The method according to claim 17, wherein the non-diffractive intraocular lens implant is a monofocal intraocular lens implant.

19. The method according to claim 17, wherein the non-diffractive intraocular lens implant incorporates 0.2 μm of negative spherical aberration.

20. The method according to claim 17, wherein the non-diffractive intraocular lens implant incorporates 0.3 μm of coma.
